(19) United States
(12) Patent Application Publication
(10) Pub. No.: US 2026/0367403 A1
(43) Pub. Date: Nov. 19, 2026

(54) METHOD AND PROTECTIVE GOGGLES FOR ASSISTING MEDICAL AESTHETIC TREATMENT

(71) Applicant: Nanning FuLian FuGui Precision Industrial Co., Ltd., Nanning (CN)

(72) Inventors: PO-HSUN HUANG, New Taipei (TW); HAU-LUNG HUANG, New Taipei (TW)

(21) Appl. No.: 19/211,503

(22) Filed: May 19, 2025

(51) Int. Cl. G02B 27/01; A61B 18/00; A61B 18/20

(52) U.S. Cl. CPC G02B 27/0172; A61B 18/20; A61B 2018/00452; A61B 2018/00642; A61B 2018/00702; A61B 2018/202; G02B 2027/0138; G02B 2027/014

(57) ABSTRACT

A method for assisting medical aesthetic treatment and protective goggles, the method including: obtaining a real scene image containing a skin image, captured by a camera module mounted on the protective goggles, dividing the skin image into a plurality of treatment areas, performing skin condition analysis and feature point identification on each area respectively, calculating an energy density corresponding to each treatment area according to the analysis results, generating a virtual image containing the treatment areas, feature points, and energy densities based on this information, superimposing it onto the real scene image to form an auxiliary medical aesthetic interface, and finally, presenting the interface through a display module of the protective goggles.

### FIELD

A method and protective goggles for assisting medical aesthetic treatment.

### BACKGROUND

In the existing medical aesthetic industry, when performing laser-related procedures (such as spot removal, hair removal, etc.), operators usually need to wear protective goggles to prevent strong light generated by the laser from causing damage to their eyes. However, traditional protective goggles, in order to block strong light, typically have high light-blocking properties, making it difficult for operators to clearly observe the treatment area, such as the location of spots or hair, during the procedure. To accurately determine the treatment area, operators often need to frequently take off their protective goggles for observation and then put them back on for operation, which not only reduces operational efficiency but also increases the risk during the surgical process.

Furthermore, due to the inability to observe the skin surface condition in real time, operators mostly rely on experience and intuition to determine whether a certain area has been irradiated by the laser, which easily leads to repeated treatments or missed spots, affecting the treatment effect.

### BRIEF DESCRIPTION OF THE DRAWINGS

Implementations of the present technology will now be described, by way of example only, with reference to the attached figures, wherein:

FIG. 1 is a schematic diagram of protective goggles for assisting medical aesthetic treatment according to one embodiment of the present disclosure.

FIG. 2 is a flow chart of a method for assisting medical aesthetic treatment according to one embodiment of the present disclosure.

FIG. 3 is a flow chart of a method for assisting medical aesthetic treatment according to another embodiment of the present disclosure.

FIG. 4 is a schematic diagram of an auxiliary medical aesthetic interface according to one embodiment of the present invention.

FIG. 5 is a hardware block diagram of protective goggles for assisting medical aesthetic treatment according to one embodiment of the present invention.

### DETAILED DESCRIPTION

It should be understood that the detailed description and specific examples, while indicating exemplary embodiments, are projected for purposes of illustration only and are not projected to limit the scope of the claims.

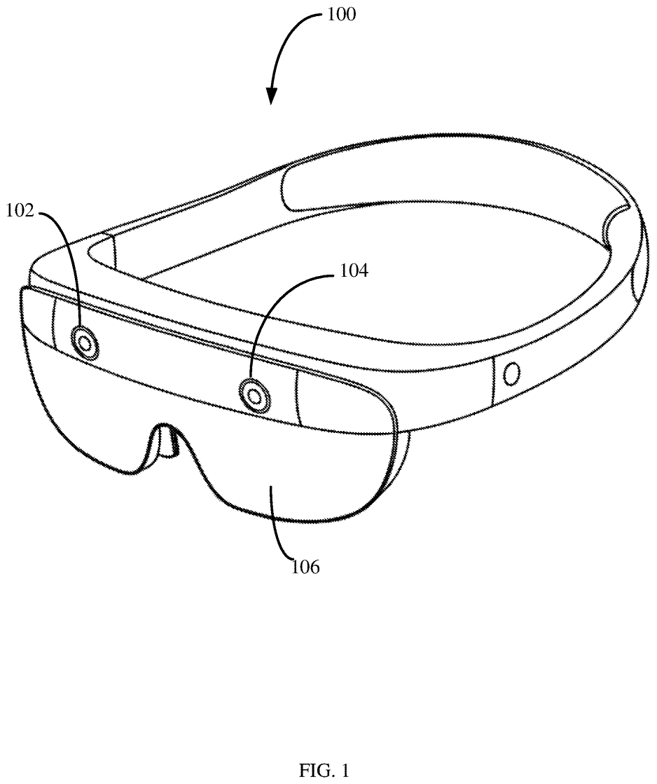

FIG. 1 is a schematic diagram of protective goggles 100 for assisting medical aesthetic treatment. It is to be understood that the protective goggles shown in FIG. 1 are merely illustrative and do not impose any limitations on the present invention. The protective goggles 100 include miniature cameras 102, 104 and an imaging mirror 106. The protective goggles 100 have an overall sunglasses appearance, and the miniature cameras 102, 104 are disposed on the frame.

What the operator sees when wearing the protective goggles 100 is not a completely real scene, but rather an image captured by the miniature cameras 102, 104 and post-processed, and the post-processed image is displayed to the operator by the imaging mirror 106.

In one embodiment, the imaging mirror 106 can simultaneously display the originally captured image and the post-processed image, which is convenient for the operator to compare and determine.

In one embodiment, the imaging mirror 106 may adopt transparent display technology, such as Micro-OLED or Micro-LED, to project the post-processed image into the operator's field of view.

In one embodiment, the imaging mirror 106 includes a light sensor and an electrochromic material. When strong light such as a laser is sensed, the electrochromic material changes its state, causing the mirror to become opaque, thereby protecting the operator's eyes.

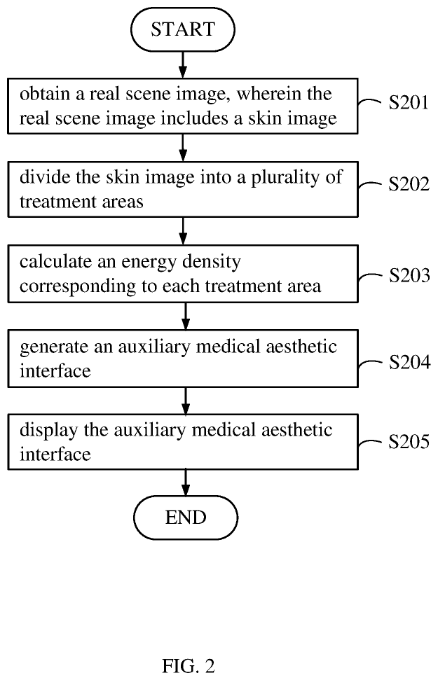

FIG. 2 is a flowchart of a method for assisting medical aesthetic treatment. The method is executed in protective goggles.

Step S201, obtain a real scene image captured by the camera module mounted on the protective goggles, wherein the real scene image includes a skin image.

When the operator wears the protective goggles, the camera module mounted on the protective goggles can capture a real scene image directly in front of the operator in real time. The protective goggles can be referred to the schematic diagram in FIG. 1. The camera module includes dual miniature cameras disposed on the front frame of the protective goggles, so that a real scene image directly in front of the operator can be captured.

In this embodiment, the operator can wear the protective goggles and face the skin area of a patient to be treated with medical aesthetics to obtain a skin image.

Step S202, divide the skin image into a plurality of treatment areas, and perform skin condition analysis and feature point identification on each treatment area respectively. The skin condition analysis includes skin color recognition and skin texture analysis. The feature points include spots and body hair.

In one embodiment, the size of the treatment area can be pre-set by the operator according to a basic unit of a medical aesthetic project, and the size of each treatment area is consistent.

Before the skin condition analysis, the skin image can be first converted from RGB to a color space that is easier to separate skin colors, such as HSV, YCbCr, or Lab.

Taking the Lab color space as an example, its color space information (L, a, b three channels) can be used to quantitatively represent the hue, brightness, and color deviation of skin with different skin tone levels, and then combined with glossiness and texture features to evaluate the skin texture condition.

For example, skin tone levels are pre-defined as 1 to 5 from dark to light, and each skin tone level corresponds to a different Lab color value. The average Lab color value of all pixels in the treatment area is taken as the skin color value of the treatment area, and then the corresponding skin tone level is determined according to the skin color value.

Dry skin usually has a rougher texture and clear pores; while oily skin has larger but smoother pores, and the overall surface has strong reflection. High-gloss ratio or local brightness values can be used to evaluate the degree of oiliness, combined with texture features to analyze whether the skin texture of the treatment area is dry skin or oily skin.

In one embodiment, since feature points such as spots and body hair have different shapes, edges, and color features in the skin image, they can be regarded as different image objects for identification. For example, body hair usually appears as slender, dark, irregular lines, while spots are usually round or irregular and have a color different from the skin tone. Feature point identification can be performed using existing known image processing methods, or it can be identified using a trained deep learning model, which will not be described in detail here.

Step S203, calculate an energy density corresponding to each treatment area according to the skin condition and feature points of each treatment area.

In one embodiment, the presence or absence of body hair and spots can be represented by T₁ and T₂ respectively. When T₁=2, it indicates that there is body hair in the treatment area. When T₁=0, it indicates that there is no body hair in the treatment area. When T₂=1, it indicates that there are spots in the treatment area. When T₂=0, it indicates that there are no spots in the treatment area. SCS (Skin Color Scale) is the skin tone level, and the value of SCS from 1 to 5 represents dark to light skin. SP (Skin Properties) is the skin characteristic, the value of SP is 1 for dry skin, and the value of SP is 2 for oily skin. Then, the energy density (ED) of each treatment area can be calculated using the following formula: ED=R((T₁+T₂)×(SCS×W_{scs}+SP×W_{SP})), where the R function is used to take the rounded value, W_{scs} is the preset SCS weighting coefficient, W_{SP} is the preset SP weighting coefficient. The W_{scs} and W_{SP} can be preset by the operator or set with reference to the setting manual of the medical aesthetic equipment. The unit of ED is the amount of energy received by the skin per treatment area.

For example, when a treatment area contains spots, body hair, dark skin tone, and dry skin texture, according to the preset SCS weighting coefficient of 0.7, the preset SP weighting coefficient of 0.3, and the size of each treatment area is 1 square centimeter, the energy density of the treatment area can be calculated according to the formula, that is ED=R((1+2)×(2×0.7+1×0.3))=5 J/cm².

In one embodiment, the energy density formula can be preset, or it can be adjusted according to the setting manual of the medical aesthetic equipment and the actual medical aesthetic project or treatment course. Specifically, the representation values of T₁ and T₂, as well as the weighting coefficients of SCS and SP, can be adjusted.

Step S204, generate an auxiliary medical aesthetic interface according to the skin image, the plurality of treatment areas, the feature points, and the energy density corresponding to each treatment area.

In one embodiment, a virtual image of the plurality of treatment areas and feature points is generated according to the skin image, and the virtual image is superimposed on the real scene image to generate the auxiliary medical aesthetic interface.

Step S205, display the auxiliary medical aesthetic interface on the display module of the protective goggles.

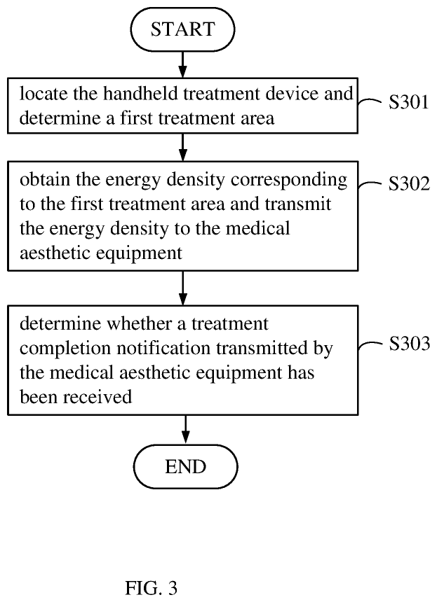

FIG. 3 is a flowchart of a method for assisting medical aesthetic treatment. The method is executed in protective goggles that are communicatively connected to a medical aesthetic equipment. The medical aesthetic equipment includes a handheld treatment device, which is a core operating end of the medical aesthetic equipment and is used to accurately transmit laser energy to and act on target skin tissue.

Step S301, locate the handheld treatment device and determine a first treatment area corresponding to the handheld treatment device.

When an operator wears the protective goggles, an auxiliary medical aesthetic interface formed by superimposing a virtual image of a plurality of treatment areas, feature points, and energy densities corresponding to each treatment area onto a real scene image can be seen.

When the operator holds the handheld treatment device close to the skin of a patient, the relative positional relationship between the handheld treatment device and the plurality of treatment areas can be observed through the auxiliary medical aesthetic interface displayed by the protective goggles. At the same time, a camera module on the protective goggles can capture a real scene image including the handheld treatment device. By analyzing the real-time captured real scene image, the first treatment area currently closest to the handheld treatment device can be located.

Step S302, obtain the energy density corresponding to the first treatment area and transmit the energy density to the medical aesthetic equipment, so that the medical aesthetic equipment sets the laser energy of the handheld treatment device according to the energy density.

In one embodiment, when the medical aesthetic equipment completes the setting of the laser energy of the handheld treatment device, it can send feedback information to the protective goggles. After receiving the feedback information, the protective goggles prompt the operator on the auxiliary medical aesthetic interface that the laser energy setting of the handheld treatment device has been completed.

At this time, the operator can press an execution button of the handheld treatment device to perform laser treatment. In response to the laser treatment execution of the handheld treatment device, the medical aesthetic equipment sends a treatment completion notification back to the protective goggles.

Step S303, determine whether a treatment completion notification transmitted by the medical aesthetic equipment has been received. When the treatment completion notification is received, the first treatment area is marked as a completed state in the auxiliary medical aesthetic interface.

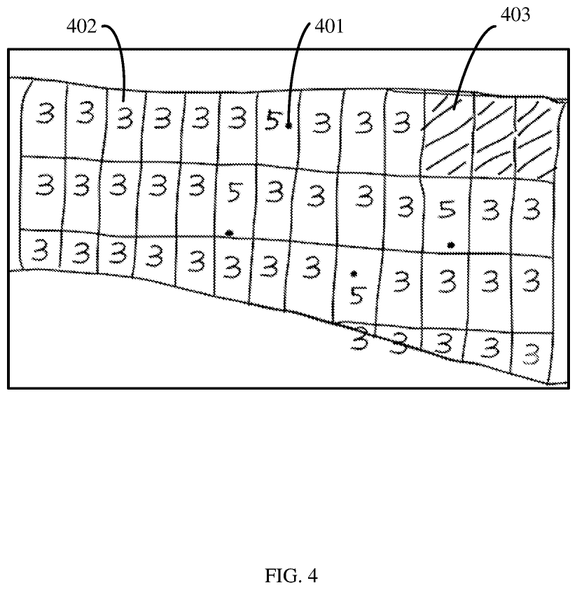

FIG. 4 is a schematic diagram of an auxiliary medical aesthetic interface. As shown in FIG. 4, the auxiliary medical aesthetic interface is a superposition of a real scene image and a virtual image. The virtual image includes a plurality of grid-like treatment areas, a feature point marker 401, an energy density marker 402, and a completed state marker 403.

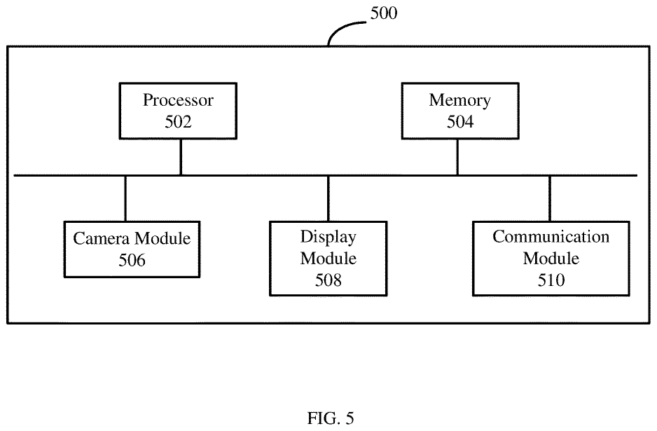

FIG. 5 is a hardware block diagram of protective goggles for assisting medical aesthetic treatment.

The protective goggles 500 include a processor 502, a memory 504, a camera module 506, a display module 508, and a communication module 510.

The processor 502 may include one or more processing cores. The processor 502 utilizes various interfaces and circuits to connect various components within the protective goggles 500. By running or executing instruction sets, computer programs, or code stored in the memory 504, and by calling data stored in the memory 504, the processor 502 performs various functions of the protective goggles 500 and processes data.

The camera module 506 is used to capture a real scene image. The display module 508 is used to display the auxiliary medical aesthetic interface.

The communication module 510 is used to communicate with external devices or systems, such as a medical aesthetic equipment. Optionally, the communication module 510 may include standard wired interfaces and wireless interfaces, such as a Bluetooth interface and a Wi-Fi interface.

In summary, the method for assisting medical aesthetic treatment and the protective goggles of the present invention allow an operator to quickly and accurately complete laser treatment through the auxiliary medical aesthetic interface while wearing the protective goggles.

It will be apparent to those skilled in the art that various modifications and variations can be made to the disclosure without departing from the scope or spirit of the claims. In view of the foregoing, it is intended that the present disclosure covers modifications and variations, provided they fall within the scope of the following claims and their equivalents.

What is claimed is:

1. A method to assist medical aesthetic treatment, executed in protective goggles, the method comprising:
obtaining a real scene image captured by a camera module mounted on the protective goggles, wherein the real scene image includes a skin image;
dividing the skin image into a plurality of treatment areas, and performing skin condition analysis and feature point identification on each treatment area respectively;
calculating an energy density corresponding to each treatment area according to the skin condition and feature points of each treatment area;
generating a virtual image of the plurality of treatment areas and the feature points and the energy density corresponding to each treatment area according to the skin image;
superimposing the virtual image onto the real scene image to generate an auxiliary medical aesthetic interface; and
displaying the auxiliary medical aesthetic interface on a display module of the protective goggles.

2. The method for assisting medical aesthetic treatment of claim 1, wherein the skin condition analysis includes: skin color recognition and skin texture analysis.

3. The method for assisting medical aesthetic treatment of claim 1, wherein the feature points include: spots and body hair.

4. The method for assisting medical aesthetic treatment of claim 1, wherein the energy density corresponding to each treatment area is calculated according to the following formula:
ED=R((T₁+T₂)×(SCS×W_{scs}+SP×W_{SP})),
wherein, ED is the energy density, the R function is used to take a rounded value, T₁ is used to indicate the presence or absence of hair, where T₁=2 indicates that there is hair in the treatment area, T₁=0 indicates that there is no hair in the treatment area, T₂ is used to indicate the presence or absence of spots, where T₂=1 indicates that there are spots in the treatment area, and T₂=0 indicates that there are no spots in the treatment area, SCS is used to indicate a skin color scale, and the value of SCS from 1 to 5 represents dark to light skin, SP is used to indicate skin properties, where SP=1 represents dry skin, and SP=2 represents oily skin, W_{scs} is a preset SCS weighting coefficient, W_{SP} and is a preset SP weighting coefficient.

5. The method for assisting medical aesthetic treatment of claim 1, wherein the display module includes an imaging mirror, the imaging mirror includes a light sensor and an electrochromic material, wherein, when the light sensor senses strong light, the electrochromic material changes its state, causing the imaging mirror to become opaque.

6. The method for assisting medical aesthetic treatment of claim 1, wherein the protective goggles are communicatively connected to a medical aesthetic equipment, the medical aesthetic equipment includes a handheld treatment device, and the method further comprises:
locating the handheld treatment device and determining a first treatment area corresponding to the handheld treatment device;
obtaining the energy density corresponding to the first treatment area;
transmitting the energy density corresponding to the first treatment area to the medical aesthetic equipment, causing the medical aesthetic equipment to set a laser energy of the handheld treatment device according to the energy density;
determining whether a treatment completion notification from the medical aesthetic equipment has been received, and when the treatment completion notification is received, marking the first treatment area as a completed state in the auxiliary medical aesthetic interface.

7. Protective goggles configured for assisting medical aesthetic treatment comprising:
a processor; and
a non-transitory memory storage comprising a computer program, wherein when the computer program is executed by the processor, the processor:
obtaining a real scene image captured by a camera module mounted on the protective goggles, wherein the real scene image includes a skin image;
dividing the skin image into a plurality of treatment areas, and performing skin condition analysis and feature point identification on each treatment area respectively;
calculating an energy density corresponding to each treatment area according to the skin condition and feature points of each treatment area;
generating a virtual image of the plurality of treatment areas and the feature points and the energy density corresponding to each treatment area according to the skin image;
superimposing the virtual image onto the real scene image to generate an auxiliary medical aesthetic interface; and
displaying the auxiliary medical aesthetic interface on a display module of the protective goggles.
